(19) United States
(12) Patent Application Publication
(10) Pub. No.: US 2018/0280509 A1
(43) Pub. Date: Oct. 4, 2018

(54) TREATMENT OF BLOOD

(71) Applicants: Robert SCHENA, Wayne, PA (US); Giana SCHENA, Wayne, PA (US)

(72) Inventors: Robert SCHENA, Wayne, PA (US); Giana SCHENA, Wayne, PA (US)

(21) Appl. No.: 15/471,611

(22) Filed: Mar. 28, 2017

(51) Int. Cl. A61K 41/00; A61K 35/14; A61K 9/00; A61N 1/44; A61M 1/36; A61M 5/158; A61M 1/10

(52) U.S. Cl. CPC A61K 41/0023; A61K 35/14; A61K 9/0019; A61M 1/1039; A61M 1/3687; A61M 1/3621; A61M 5/158; A61N 1/44

(57) ABSTRACT

Activation of senescent stem cells in humans to promote wound healing and other therapies, and to promote general enhancement of health by application of non-thermal atmospheric pressure plasma (NTAPP) to tissues and body fluids such as blood. In particular, the targets of the NTAPP are stem cells. The products contained in NTAPP can affect the behavior of peripheral blood stem cells (PBSC) in blood, as well as stem cells found in tissues surrounding the blood in which said products are carried.

### TECHNICAL FIELD

The present invention is directed to a method of treating blood, and especially to a method of activating senescent stem cells in humans to promote wound healing, and general enhancement by non-thermal atmospheric pressure plasma application to blood.

### BACKGROUND

Aging is a complex process in which a functional decline in cells and tissues results in reduced regenerative capacity of said tissues in response to injury. Adult stem cells are tissue-specific, and are capable of self-renewal in order to maintain stem cell reserves or to differentiate into various cell types. These unique properties of stem cells are essential for regenerative repair throughout the lifetime of an individual. With age, the capacity for self-renewal of stem cells declines, resulting in an accumulation of unrepaired or damaged tissues in older individuals. The loss of the cells' power of proliferation is known as senescence.

The activation of these senescent stem cells can lead to an increase in function and the promotion of healing and general health enhancement. Adult stem cells are found in small numbers in various tissues including bone marrow and adipose tissue. As the name suggests, peripheral blood stem cells (PBSC) are found in blood. Stem cells have the ability to differentiate into unrelated cell types and are not limited to the tissue in which they are found.

Studies in mice have shown that an anti-aging effect can be achieved through the injection of a protein, growth-differentiation factor 11 (GDF 11). Kaiser, “Young Blood Renews Old Mice”, www<dot>sciencemag<dot>org/news/2014/05/young-blood-renews-old-mice. May 4, 2014. GDF 11 is known to regulate stem cell activity in mice and humans and is abundant in young mice. The availability of GDF 11 decreases with age, and clinical trials have shown that injected GDF11 can restore the function of stem cells in tissues such as the heart, brain, liver, muscle, etc. GDF11 has also been found to aid in the recovery from wounds in aging animals via the growth of new blood vessels and several other mechanisms. Through further clinical trials, the use of GDF11 has become controversial and alternative methods are surfacing.

Non-Thermal Atmospheric Pressure Plasma (NTAPP) can be generated easily from gases such as helium and argon at atmospheric pressure and room temperature. The application of electricity allows the plasma to form at low temperatures, without heating. Since the temperature of the generated plasma falls below the threshold for thermal damage in cells, many potential biomedical applications have surfaced.

It is nearly impossible to detect the species present in NTAPP once it is produced, but it is known that the identity of the gas, the strength of the electric field applied, and the duration of the application of the electric field contribute to the composition of the plasma. When plasma is created from a gas such as helium in air, the most abundant species produced are atmospheric oxygen (O₂), nitric oxide (NO), and ozone (O₃). These components are known as “reactive oxygen species” (ROS) and are said to be responsible for the significant effects seen in the behavior of cells exposed to NTAPP. At basal levels, ROS produced by NTAPP, along with charged particles and UV radiation, have shown proliferative effects on eukaryotic cells. [Cite?]

In vitro exposure of porcine aortic endothelial cells provides evidence for the proliferation of cells due to NTAPP exposure. Kalghatgi, et al. Ann. Biomed. Eng. 38(3): 748-757, 2010. After thirty seconds of exposure, cells treated with NTAPP showed double the proliferation compared to untreated cells, after five days. The proliferated cells showed increased release of fibroblast growth factor 2 (FGF2), which induces cells to invade surrounding tissue, proliferate, and develop. FGF2 is known to play a crucial role in developmental events such as limb development as well.

Several applications of NTAPP are currently being explored. In order to determine its apoptotic effects on p53-mutated cancer cells, cells have been exposed to NTAPP in vitro using a dielectric barrier discharge (DBD) device. Ma, et al., PLoS ONE, 9(4): e91947, 2014.

The plasma needle and the plasma pencil (a slightly modified version) have been used to show the proliferative effects of NTAPP on several cell types in vitro. It has also been suggested that these devices have a potential for applications in dental medicine. Stoffels, et al. Plasma Sources Sci. Technol. 15: S 169-S 180, 2006.

Nitric Oxide produced from NTAPP has been applied topically in order to determine its effects on wound healing.

### SUMMARY

In one embodiment of the present invention, an apparatus is provided that exposes a portion of a patient's blood to NTAPP, while the blood otherwise remains in normal circulation.

In another embodiment, a plasma needle is used to expose blood to NTAPP in vivo.

In a further embodiment, an implanted device is used to expose blood to NTAPP at periodic intervals controlled by the patient.

An embodiment provides a process for treating blood with non-thermal atmospheric pressure plasma, comprising exposing a portion of a patient's blood to at least one of non-thermal plasma generated at atmospheric pressure and room temperature (NTAPP) and reactive oxygen species (ROS) generated by the interaction of NTAPP with oxygen-containing gas, and controlling levels of NTAPP and/or ROS to which the blood is exposed to stimulate cells in the blood and to avoid cell stress/death.

The level of NTAPP may be controlled not to exceed a basal level, which may be 8 J/cm², more preferably not to exceed 4 J/cm². In an embodiment, charged gas particles that are not ROS and UV rays always stay within the plasma jet. Therefore, a situation where the air, now containing ROS, diffuses out of the plasma jet is much safer because it only contains the ROS. Kalghatgi indicates that 8 J/cm² is an energy threshold for cell damage. Ma also provided guidance on a suitable density of ROS at different times of the treatment.

Kalghatgi does mention that ROS does sub-lethal damage to cell membranes. This renders the cell leaky to compounds contained within the cytosol, such as FGF2. However, because the cell is equipped with mechanisms for repair, and because at these dosage levels the repair mechanism is activated, so that the cells end up with increased viability and/or proliferation compared with their condition before the treatment, that is not regarded in this context as “cell stress/death.”

Also, from prior knowledge studying FGF2, the main function of that growth factor is not specifically for repair, but for proliferation. The FGF2 molecule is often identified in the early development of mice and chickens, playing an important role in limb induction. This could suggest that the FGF2 is not a response to the damage, but since it is released due to the leaky membrane, causes proliferation in surrounding tissues.

The portion of blood may be removed from the patient, exposed to the NTAPP, and returned, while a remainder of the patient's blood is in normal circulation within the patient.

The portion of blood may be removed and returned via a continuous flow path, while the blood is otherwise in normal circulation. That may be done using a device based on a modified apheresis machine having an intake line and a separate return line. That allows new blood to be drawn while the already treated blood is returned. Alternatively, a device with only one tube and a reversible pump may be used. However, the blood would then have to be drawn in batches, and each batch returned before new blood is drawn, making the process lengthier.

Alternatively, the blood may be directly exposed to NTAPP in vivo with the use of a plasma needle, or via the use of an implanted device.

In an embodiment, an apparatus for treating blood with non-thermal plasma generated at atmospheric pressure and room temperature (NTAPP) in vitro, comprises (a) a non-thermal plasma component comprising a holding area for blood, having an air space to which a liquid surface of the blood is exposed, first and second electrodes on opposite sides of the air space, at least one of said electrode insulated from the air space by a dielectric barrier, a gas delivery system for supplying ionizable gas to the air space, an alternating current (AC) power supply that in operation applies across the electrodes a voltage effective to generate a NTAPP in the ionizable gas in the air space, and a controller operative to regulate the AC power supply and the gas delivery system to generate in the air space an NTAPP at a level effective to stimulate cell development in blood in the holding area; and (b) a blood pump component to deliver blood from a patient to the holding area, and from the holding area back to the patient.

The blood pump component comprises a hypodermic needle, and a peristaltic pump connected between the hypodermic needle and the holding area.

The apparatus may further comprise a supply of helium as the ionizable gas.

The controller may further comprise at least one of a mass flow controller to regulate gas delivery and a current voltage controller to regulate the AC power supply.

The blood pump component further comprises a peristaltic pump.

The blood pump component may further comprise a hypodermic needle, the blood pump component then being connected to pump blood between the hypodermic needle and the holding area.

In another embodiment, an apparatus for treating blood with NTAPP in vivo comprises (a) a device to be implanted into a patient, said device comprising a dielectric barrier discharge (DBD) plasma generator; (b) a wireless power component to be implanted into the patient to provide power to the DBD plasma generator in a controlled manner, said wireless power component being remotely accessible by computer for configuration and programming of said component; and (c) a tube that extends from the DBD plasma generator to the surface of the skin for gas input.

Other embodiments provide combinations of features of two or more of the embodiments mentioned above or described below.

### DESCRIPTION OF THE DRAWINGS

While the appended claims set forth the features of the present techniques with particularity, these techniques, together with their objects and advantages, may be best understood from the following detailed description taken in conjunction with the accompanying drawings, in which like numerals refer to like parts.

FIG. 1 is a schematic view of a first apparatus that may be used to expose a patient's blood to NTAPP, while the blood otherwise remains in normal circulation.

FIG. 2 is a diagram of a dielectric barrier discharge device (DBD) device forming part of the apparatus shown in FIG. 1.

FIG. 3 is a schematic view of a second apparatus, using a plasma needle.

FIG. 4 is a view of a third apparatus, to be implanted into a patient.

FIG. 5 is a top/side view of a plastic sheet forming part of the apparatus of FIG. 4.

### DETAILED DESCRIPTION

Turning to the drawings, wherein like reference numerals refer to like elements, techniques of the present disclosure are illustrated as being implemented in a suitable environment. The following description is based on embodiments of the claims and should not be taken as limiting the claims with regard to alternative embodiments that are not explicitly described herein.

It is to be understood that the FIGS. and descriptions herein are simplified to illustrate elements that are relevant for a clear understanding of the disclosure, while eliminating, for the purpose of clarity, many other elements known in the art. Those of ordinary skill in the art may recognize that other elements and/or steps are desirable and/or required in implementing the disclosed embodiments. Elements and steps well known in the art, or that do not facilitate a better understanding of the disclosure, are not disclosed. The disclosure is directed to variations and modifications to disclosed elements and methods that will be known to those skilled in the art.

As envisioned in the present invention with respect to the disclosed compositions of matter and methods, in one aspect embodiments of the invention comprise the components and/or steps disclosed herein. In another aspect, embodiments of the invention consist essentially of the components and/or steps disclosed herein. In yet another aspect, embodiments of the invention consist of the components and/or steps disclosed herein.

ROS may be introduced into the blood (extracellular) at high enough concentration to provide for the triggering of intracellular ROS-sensitive signaling cascades. These cascades are believed to result in similar “gain-of function” effects shown by the injection of exogenous GDF11, including angiogenesis and improved organ function due to cell proliferation.

One specific type of stem cells, peripheral blood stem cells (PBSC), is present in blood. When exposed to Non-Thermal Atmospheric Pressure Plasma (NTAPP), they may be induced to produce signaling molecules such as GDF11 that can improve health in a similar fashion to the benefits observed in the exogenous administration of GDF 1. The “new” blood would eventually enter the bone marrow, where the contained ROS and perhaps signaling molecules from PBSCs can induce mesenchymal stem cells. These stem cells have the ability to differentiate into osteoblasts, chondrocytes, adipocytes, tenocytes, etc. Adult stem cells are located in various tissues through which blood flows including the brain, skeletal muscle, blood vessels, the liver, and several other sites.

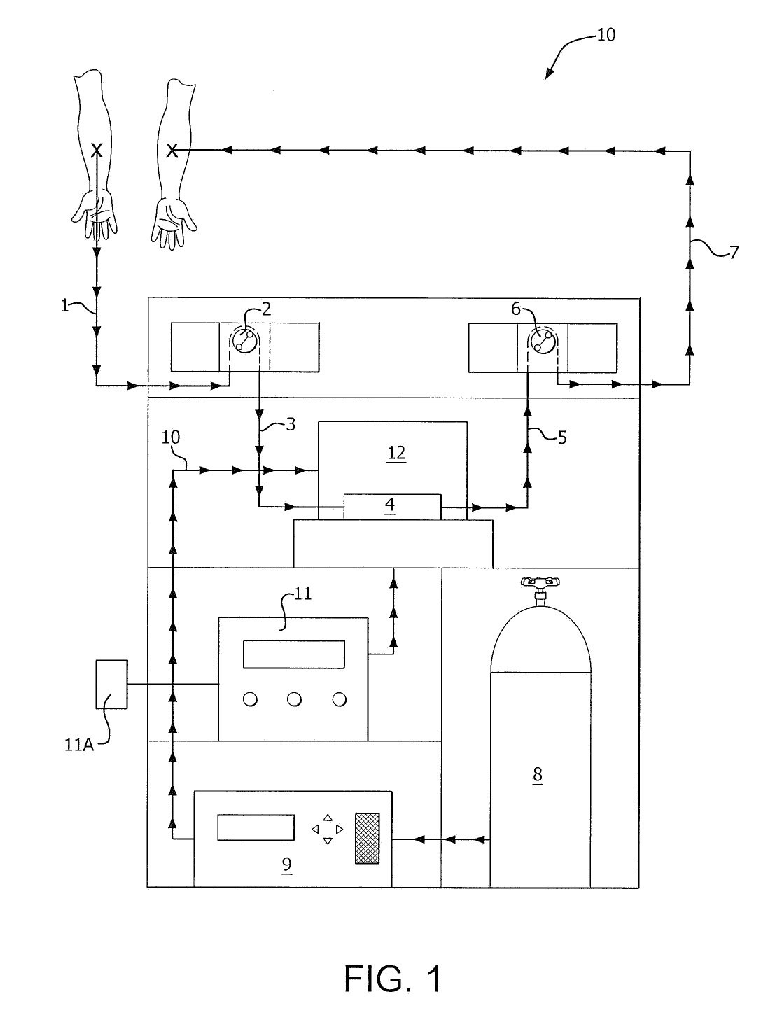

Referring to the drawings, FIG. 1 is a schematic view of a first embodiment of an apparatus 10 that may be used to expose a patient's blood to NTAPP, while the blood otherwise remains in normal circulation. A top portion of the apparatus contains blood pumps 2, 6 to bring blood from the patient to a holding area 4, and to send blood from the holding area back to the patient. The blood handling part of the system may be similar to an apheresis type blood pump. A lower portion of the apparatus contains a dielectric barrier discharge device (DBD) 12 surrounding the holding area 4. The DBD 12 is connected to a helium supply 8, a flow controller, and a voltage controller. The main idea is that a portion of blood will be removed from the patient, exposed to NTAPP, and returned back to the patient while the blood is otherwise in normal circulation. In order for the blood sample to be exposed to NTAPP, it must be pumped from the patient into the holding area 4, which in this embodiment resembles a modified petri dish or a container of similar size. The holding area is located inside the DBD 12, where the NTAPP will be generated. The first blood pump 2 will transfer blood from the patient via a blood withdrawal line 1, and along to the holding area 4 via a continuation 3 of the withdrawal line 1. A second blood pump 6 will transfer blood from the holding area 4 via a return line 5, and back to the patient through an extension 7 of the return line 5.

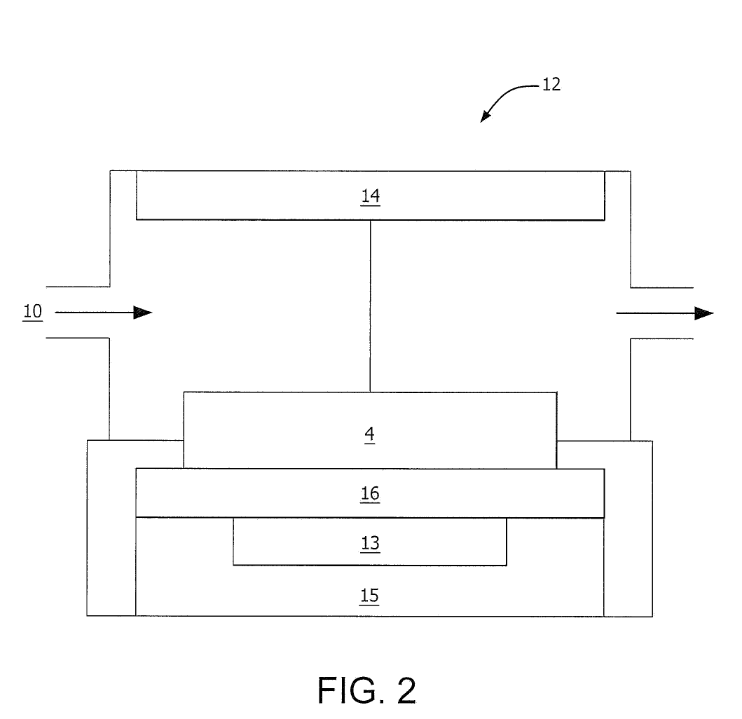

Referring also to FIG. 2, under the blood sample, within the DBD chamber 12, sits a grounded electrode 13. A layer of dielectric material 16 covers the electrode 13 and an insulating material 15 in which the electrode 13 is embedded. Another electrode 14 of the same material sits above the blood sample holding area 4 and may remain completely exposed. Past trials, see Ma, et al., PLoS ONE, 9(4): e91947, 2014, have had success in using copper electrodes 13, 14 with polytetrafluoroethylene (PTFE) as the dielectric material 16. The thickness of the PTFE layer can be used as a control to limit the amount of charge produced between the two electrodes. It would be necessary to coordinate the thickness with the applied voltage to obtain the desired charge.

An alternate current (AC) power supplier 11 will apply a voltage to the electrodes 13, 14, creating an electric field between the two electrodes. The power supplier 11 will also serve as a controller. In an embodiment, the incoming power supply to the power supplier 11 is low-voltage direct current (DC), and the DC voltage can be varied using a controller 11A to determine the AC voltage applied to the electrodes. A gas-feeding system 8, 9, 10 feeds a supply of helium 8 or other suitable gas through a mass flow controller 9 and into the DBD chamber 12 via a supply line 10.

The helium flowing between the two electrodes 13, 14 will be influenced by the applied current to generate NTAPP, plasma at substantially room temperature and atmospheric pressure. The gas will flow perpendicular to the electric field between the two electrodes to ensure that the reactive oxygen species (ROS) formed can diffuse while the charged particles produced stay within the plasma jet. Ma reported that in his device He gas flowed into the inlet of the device at a rate of 1 to 5 standard liters per minute (SLM). 0.1 ms was estimated as the arrival time of the ROS species from the plasma nozzle to the dish. The total mole number can be estimated by multiplying the surface area of the dish and total amount of interaction time. For example, with a dish diameter of 3.5 cm and with operation times of 30 seconds, the total multiplication factor is 289. Therefore, the maximum mole number of ozone delivered to the media is approximately 30 mmol. These parameters, scaled for the dimensions of a specific DBD chamber, can serve as guidelines as to how much plasma can be produced.

These charged particles can rupture the membranes of bacterial cells, resulting in a sterilization effect. All components of the DBD component 12 that are exposed to the plasma may be covered by glass or other suitable unreactive material so the diffusion of the ROS is confined to the area of the blood sample. The AC electricity applied may be in the range of hundreds of volts to a few kilovolts, depending on the distance between the two electrodes 14, 16, while the AC frequency of the pulses administered may fall within a range of one kilohertz (kHz) to ten megahertz (MHz). The voltage, current, frequency, duty cycle, and number of pulses of the applied electricity, the choice of gas, and the gas flow rate, can be altered to fit the needs of each patient, and are determined empirically in dependence on the geometry of the DBD unit 12. Some earlier experimental results indicate that in living cells, the best results may appear when cultures are subjected to NTAPP for thirty seconds each hour, for up to nine hours (or ten exposures), followed by fifteen hours of incubation. This is true; however, Ma, FIGS. 2C and 2E, showed that when these exposure conditions were applied to IMR90 cells (fibroblasts) and ASC (adipose-derived stem cells), cell proliferation was increased. The relative percentages of viable cells in ASC and IMR90 after NTAPP treatment were 178% and 168% respectively, compared to 150% for untreated cells. Kalghatgi et al. also reports that porcine endothelial cells exposed to 30 s of NTAPP showed twice the proliferation compared to untreated cells 5 days after treatment. NTAPP was toxic after 60 s, suggesting the 30 s limit. At 30 s the dosage was 4 J/cm² plasma discharge power density, and at 60 s the dosage was at 8 J/cm². Kalghatgi estimates that between 0.3 and 0.5 ROS are generated for every electron volt of plasma energy (1 eV=1.609×10⁻¹⁹ J), corresponding to 7.32×10¹⁶ to 1.22×10¹⁷ ROS at 3.9 J/cm² (implying about 7.5×10¹⁶ to 1.25×10¹⁷ ROS/cm² at 4 J/cm²). These numbers could be good guidelines to ensure the ROS concentration is not toxic along with the supplied current, etc.

Since the concept is to return the blood into circulation, incubation will not occur, or will occur in vivo after the blood is returned to circulation, and the duration of exposure can be varied on situational basis. Once the sample has been treated with the generated NTAPP, the blood will be pumped back to the patient by the pump 6, and enter normal circulation.

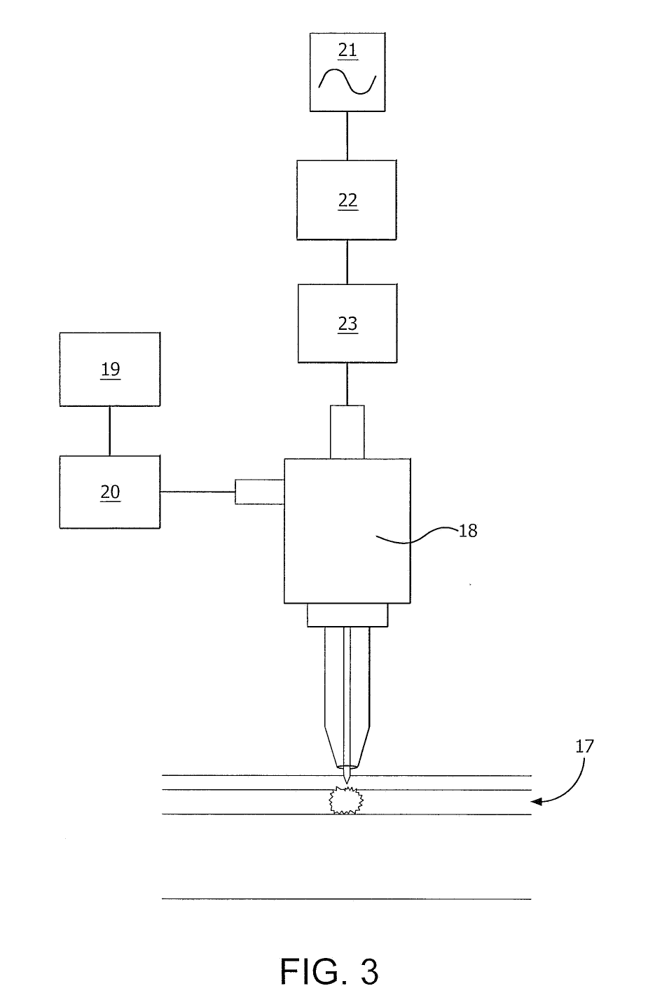

Referring now to FIG. 3, another embodiment enables the use of an already existing NTAPP delivering device. Recently, plasma needles and plasma pencils (a modified plasma needle) have been used to expose cells to NTAPP in vivo. This allows operation to remain under the threshold of thermal damage, with the goal to chemically induce a specific response or modification of different cell types. Again, the radicals generated in the plasma are responsible for the effect on cell behavior. Keeping in mind the basic concept presented in the study regarding mice and GDF11 (see “Background”, above), the same idea may be applied; however, unlike the method described above, the plasma is delivered directly into the blood 17.

A mass flow controller 20 (A suitable controller for use with currently commercially available plasma needles is the Brooks series 5850E controller from Brooks Instrument, LLC) placed between the helium supply 19 and the plasma needle 18 will monitor gas flow. A radio-frequency (RF) waveform generator 21 and an amplifier 23 will provide the power needed to generate plasma. A voltage controller 22 placed between the amplifier and the RF power source will monitor the applied power. The tip of the plasma needle 18 will be directly inserted into a blood vessel 17 near the surface of the skin. As noted above, it may be desirable for the blood to be exposed to the plasma for about thirty seconds per hour, definitely less than sixty seconds per hour, to prevent the induction of stress responses in the cells. However, where the blood in circulation is flowing past the plasma needle 18, each part of the blood is exposed to the NTAPP treatment for only a short period, and only a small amount of blood is exposed to the NTAPP treatment at any given time, so a more prolonged but less intense cycle of operation of the plasma needle 18 may be appropriate. Alternatively, a regime in which the exposure as the blood passes the plasma needle is limited based on the 4 J/cm² dose suggested by Kalghatgi's results may be appropriate.

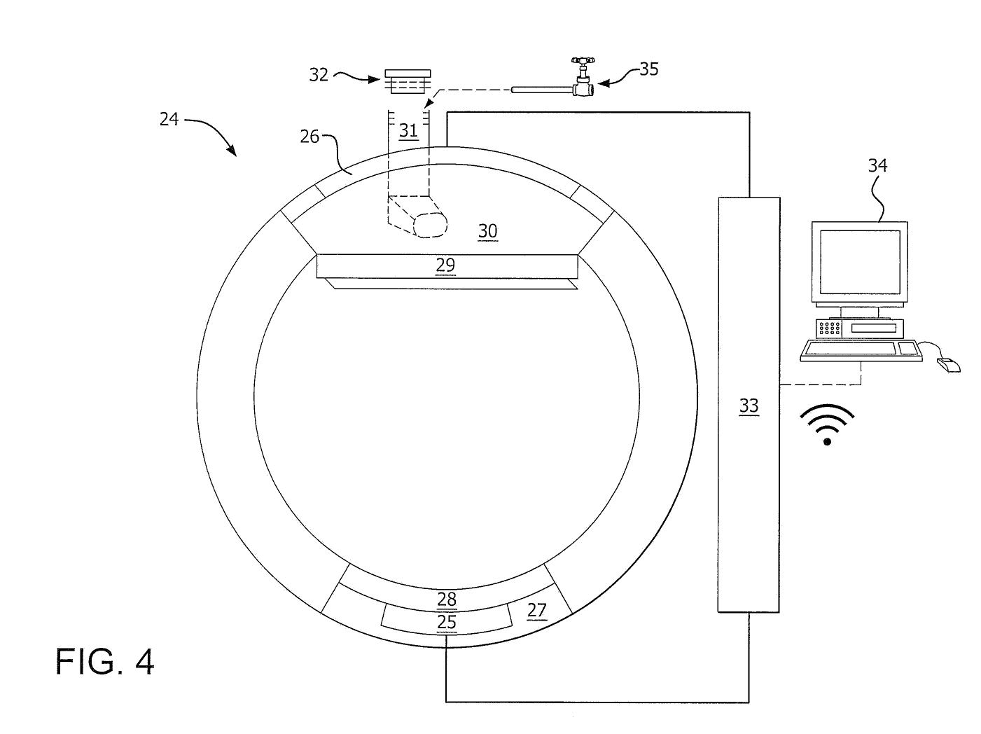

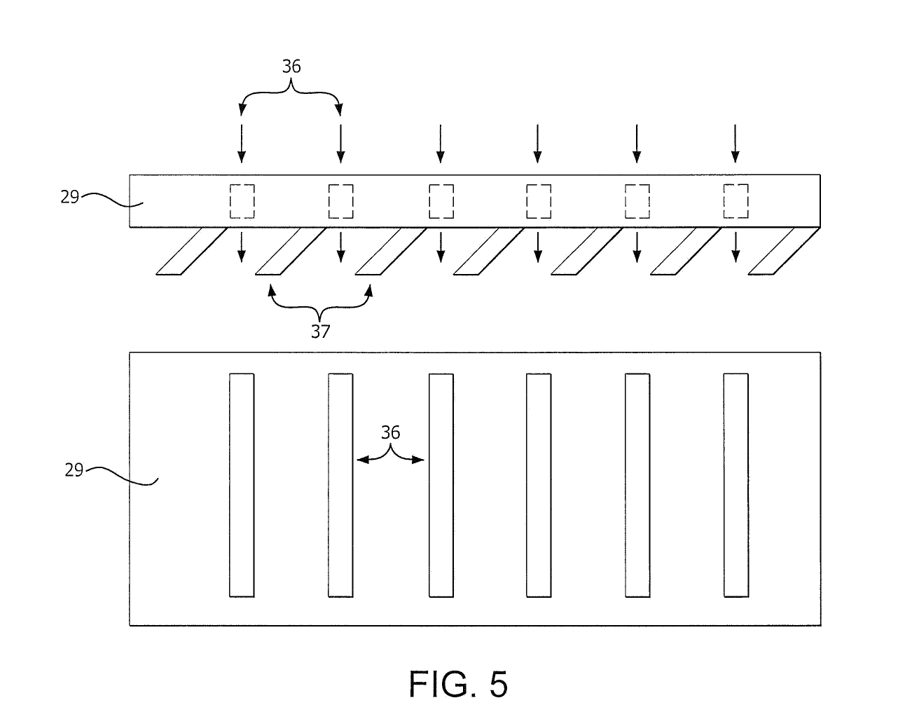

Referring to FIGS. 4 and 5, in a third embodiment. NTAPP is applied to blood by use of a subcutaneously implanted device 24. The device 24 resembles a small-scale DBD device and is implanted directly into the circulatory system in an easily accessible portion of the body, such as the forearm. The device 24 may be tube-shaped, containing an electrode 26 in the upper portion of the tube, and another electrode 25 in the bottom portion of the tube. The lower electrode 25 is grounded in an insulating material 27 and covered by a dielectric barrier 28, while the upper electrode 26 is exposed. A sheet of plastic 29 spans across the tube towards the upper portion, to create an air space 30 in which the NTAPP forms. The upper electrode 26 is exposed to the air space 30. The plastic sheet 29 may contain small slits 36 and small extensions 37 angled with the direction of blood flow to allow the blood to come in contact with the NTAPP without entering the air space 30. The air space 30 is closed off at each end of the tube, again to prevent blood from flowing through. The plastic sheet 29 may be made from a non-conducting material so the electric field can pass freely between the two electrodes 25, 26.

The air space 30 may be supplied with helium gas flowing from an inlet tube 31 spanning from the device 24 to the surface of the skin to an outlet tube (not shown) symmetrically positioned at the other end of the air space 30. Mechanisms to provide a suitable flow of ionizable helium and oxygen or clean air to provide oxygen for the ROS are well known, and in the interests of conciseness the gas supply manifold is not described in more detail. The gas supply 35 includes a small helium delivery tube that can be placed inside of the inlet tube 31, and is monitored by the user to control gas flow. The helium tube 31 is capped 32 to prevent air from entering while the device 24 is not in use. Helium will enter the device in a direction perpendicular to that of the electric field so the products of the NTAPP can diffuse along the length of the device 24. The electrical power for the DBD may be provided by a small, wireless, battery-powered device 33 that delivers pulses of electricity in a manner similar to a wireless pacemaker. The device 33 may be an implanted battery powered unit, and may be a wireless device recharged by electrical induction. US Patent Application No. 2014/0084858 A (Kim et al., assigned to Samsung Electronics Co., Ltd.) proposes an example of a wireless charging system for medical devices that may be suitable. Alternatively, the device 33 may be provided with wired electrical connections alongside the gas supply tube 31. If a wired connection is used, then a combined connector, in which all gas and electrical connections are made in a single action, and all are released in a single action, is preferred in order to simplify operation.

The power supply device 33 can be accessed via a computer 34 through either a wired or a wireless connection to turn the power on or off and to monitor power output during use. WiFi technology is currently believed to be suitable. The device 24 is implanted in a manner similar to the delivery of a stent during an angioplasty.

Because the blood space and the air space 30 of the device 24 may be considerably smaller than those of the device 12 shown in FIGS. 1-2, the helium flow, and the voltage, current, frequency, and duty cycle of the AC electrical supply, are adjusted appropriately.

As used herein, “non-thermal” or “non-thermal atmospheric pressure plasma” is a partially ionized gas ignited at atmospheric pressure and room temperature by adding energy to the gas. In the cases presented, electricity serves as the energy source. The plasma that is generated typically has a temperature of about 30° C. to 40° C., in order to treat living cells and tissues below the threshold for thermal damage. Other suitable sources of non-thermal plasma include plasma jets, corona discharge plasma sources, and dielectric barrier discharge plasma sources.

For the purposes of promoting an understanding of the principles of the invention, reference has been made to the embodiments illustrated in the drawings, and specific language has been used to describe these embodiments. However, this specific language intends no limitation of the scope of the invention, and the invention should be construed to encompass all embodiments that would normally occur to one of ordinary skill in the art. The terminology used herein is for the purpose of describing the particular embodiments and is not intended to be limiting of exemplary embodiments of the invention. In the description of the embodiments, certain detailed explanations of related art are omitted when it is deemed that they may unnecessarily obscure the essence of the invention.

The use of any and all examples, or exemplary language (e.g., “such as”) provided herein, is intended merely to better illuminate the invention and does not pose a limitation on the scope of the invention unless otherwise claimed. Numerous modifications and adaptations will be readily apparent to those of ordinary skill in this art without departing from the spirit and scope of the invention as defined by the following claims. Therefore, the scope of the invention is defined not by the detailed description of the invention but by the following claims, and all differences within the scope will be construed as being included in the invention.

No item or component is essential to the practice of the invention unless the element is specifically described as “essential” or “critical.” It will also be recognized that the terms “comprises,” “comprising,” “includes.” “including,” “has,” and “having,” as used herein, are specifically intended to be read as open-ended terms of art. The use of the terms “a” and “an” and “the” and similar referents in the context of describing the invention (especially in the context of the following claims) are to be construed to cover both the singular and plural, unless the context clearly indicates otherwise. In addition, it should be understood that although the terms “first,” “second,” etc. may be used herein to describe various elements, these elements should not be limited by these terms, which are only used to distinguish one element from another. Furthermore, recitation of ranges of values herein are merely intended to serve as a shorthand method of referring individually to each separate value falling within the range, unless otherwise indicated herein, and each separate value is incorporated into the specification as if it were individually recited herein.

1. A process for treating blood with non-thermal atmospheric pressure plasma, comprising:
exposing a portion of a patient's blood to at least one of non-thermal plasma generated at atmospheric pressure and room temperature (NTAPP) in the apparatus of claim 8 and reactive oxygen species (ROS) generated by the interaction of NTAPP with oxygen-containing gas;
controlling levels of NTAPP and/or ROS to which the blood is exposed to stimulate cells in the blood and to avoid cell stress/death.

2. A process as in claim 1, wherein the level of NTAPP does not exceed 8 J/cm².

3. A process as in claim 2, wherein the level of NTAPP does not exceed 4 J/cm².

4. A process as in claim 1, wherein the portion of blood is removed from the patient, exposed to the NTAPP, and returned, while a remainder of the patient's blood is in normal circulation within the patient.

5. A process as in claim 4, wherein the portion of blood is removed and returned via a continuous flow path.

6. A process as in claim 1, wherein the blood is directly exposed to NTAPP in vivo with the use of a plasma needle.

7. A process as in claim 1, wherein blood is treated with NTAPP via the use of an implanted device.

8. Apparatus for treating blood with non-thermal plasma generated at atmospheric pressure and room temperature (NTAPP) in vitro, comprising:
(a) a non-thermal plasma component comprising:
a holding area for blood, having an air space to which a liquid surface of the blood is exposed;
first and second electrodes on opposite sides of the air space, at least one of said electrode insulated from the air space by a dielectric barrier;
a gas delivery system for supplying ionizable gas to the air space;
an alternating current (AC) power supply that in operation applies across the electrodes a voltage effective to generate a NTAPP in the ionizable gas in the air space; and
a controller operative to regulate the AC power supply and the gas delivery system to generate in the air space an NTAPP at a level effective to stimulate cell development in blood in the holding area;
(b) a blood pump component to deliver blood from a patient to the holding area, and from the holding area back to the patient.

9. An apparatus according to claim 8, wherein the blood pump component comprises a hypodermic needle, and a peristaltic pump connected between the hypodermic needle and the holding area.

10. An apparatus according to claim 8, further comprising a supply of helium as the ionizable gas.

11. An apparatus according to claim 8, wherein the controller further comprises at least one of a mass flow controller to regulate gas delivery and a current voltage controller to regulate the AC power supply.

12. An apparatus according to claim 8, wherein said blood pump component further comprises a peristaltic pump.

13. An apparatus according to claim 8, wherein said blood pump component further comprises a hypodermic needle, and the blood pump component is connected to pump blood between the hypodermic needle and the holding area.

14. Apparatus for treating blood with NTAPP in vivo, comprising:
(a) a device to be implanted into a patient, said device comprising a dielectric barrier discharge (DBD) plasma generator;
(b) a wireless power component to be implanted into the patient to provide power to the DBD plasma generator in a controlled manner, said wireless power component being remotely accessible by computer for configuration and programming of said component; and
(c) a tube that extends from the DBD plasma generator to the surface of the skin for gas input.
